(19) United States
(12) Patent Application Publication
(10) Pub. No.: US 2007/0083110 A1
(43) Pub. Date: Apr. 12, 2007

(54) Programmable phase velocity in an ultrasonic imaging system

(75) Inventors: Shengtz Lin, Cupertino, CA (US); Tony Wu, San Jose, CA (US); Phung Hue, Cupertino, CA (US)

Correspondence Address: Bo-In Lin, 13445 Mandoli Drive, Los Altos Hills, CA, 94022, US

(73) Assignee: SonoWise, Inc.

(21) Appl. No.: 11/246,731

(22) Filed: Oct. 9, 2005

(51) Int. Cl. A61B 8/00

(52) U.S. Cl. 600/437

(57) ABSTRACT

An ultrasonic image scanning system for scanning an organic object includes a beam former that provides a phase velocity adjustment function for producing an ultrasonic image with a programmable phase velocity. The ultrasonic image scanning system further includes a beam profile analysis function for calculating an optimal phase velocity with a user controller to adjust the phase velocity until a scan image of best image quality is achieved. Alternately, the system may provide an automatic phase velocity-scanning controller for automatically scanning through a range of phase velocities and selecting a best phase velocity generating a scanning image of a best quality. The system further includes a region of interest (ROI) controller for a user to select a region for scanning with a specific focal area for optimizing the phase velocity. The system may further provide a maximum gradient analyzer for selecting an image of a best quality in optimizing the phase velocity. A digital controller may also provide a real time programmable control by applying different control algorithms with combination of phase velocity and attenuation adjustment. A hardness computational processor is implemented to determine a tissue hardness using the phase velocity and in combination with the attenuation parameter.

### FIELD OF THE INVENTION

This invention generally relates to system and method for carrying out a medical imaging process. More particularly, this invention relates to an ultrasonic imaging apparatus and method for providing the tissue hardness information through the image focus discrimination when those images are acquired by applying various phase velocities in the beam forming calculation.

### BACKGROUND OF THE INVENTION

Even though the hardness of a biological tissue is an important parameter for detection of abnormal tissue growth to enable an early detection of diseases occurring to a biological organ and greatly improve the curing rates, there are still technical difficulties and limitations faced by those applying ultrasonic imaging technologies for accurately detect biological tissues of different hardness. Specifically, conventional technologies have attempted to obtain a hardness profile of an ultrasonic scanned image by assuming that the phase velocity, i.e. speed of sound in the media, of an ultrasonic wave is constant at 1540 m/s. With this assumption, different techniques have been applied for hardness profile measurements. However, several of these measurements can only obtain hardness profile measurement with limited accuracies.

Specifically, measurements of some conventional techniques attempted to obtain the hardness profile by measuring attenuation of ultrasonic transmission in a biological organ to detect different hardness in a biologic organ as that published in an article “Broadband ultrasound attenuation” by General Electric submitted through the Information Disclosure Statement (IDS) together with the Patent Application. However, there are no consistently determinable functional relationships between the attenuation of the ultrasonic transmission through the biological tissues of different values of tissue hardness. The attenuation may be due to the tissue absorption of the energy, and not necessary to be the hardness. Additionally, there are operator's errors such as the Time Gain Compensation (TGC), Focus point and Aperture size settings, etc. can also affect the attenuation, which discounts the accountability of the tissue hardness characterization. For these reasons, the measurements of attenuations do not usually provide reliable indications of the tissue hardness and the hardness profile measurements therefore have only limited usefulness.

The propagation phase velocity changes when the ultrasound travels in the soft tissue with different hardness. The phase velocity increases with the increase of the hardness of the wave transmission media. By detecting a portion of tissue with different hardness, a soft tissue disease for example the liver cirrhosis, breast mass, prostate tumor or uterus fibroid can be evaluated without biopsy. Such detection process is very desirable especially during the treatment, when the non-invasive measurement is preferred. Hardness evaluation is performed according to the ultrasound physics, sound wave reflects echo when the media has non-uniformity, and travels at different speed when media density different because the higher is the velocity when the wave is transmitted in the medium that has higher density (or hardness). Since an ultrasound imaging system detects the reflected echo for hardness measurement, conventional technologies often measure the attenuation as a parameter to determine the non-uniformity of hardness in a soft tissue. As shown in FIGS. 1A and 1B, the hardness can be evaluated by measuring the attenuation along the propagation paths. However, as discussed above, the functional relationship between the hardness profile and the attenuation are changed under different measurement circumstances including but not limited to operators' skills and possible errors, the accuracies of measurements based on attenuation measurements are therefore degraded and hasn't been successful in the commercial available ultrasound imaging system.

FIGS. 1A and 1B, are images of echo signals as illustrations of a prior art scanning system. The 1A image has higher attenuation (0.68 db/MHz/cm) and 1B has lower tissue attenuation (0.47 dB/MHz/cm). The images are reverse in video from the conventional ultrasound images to make it more visible on the paper. As shown in these figures and further plotted in FIG. 1C, the 0.68 dB image attenuated faster than the 0.47 dB with both tissues at 1543 m/s. The user can set a vertical line on the image, and the system can take the image data out of the line and plot a curve versus depth. With all the TGC, focal point etc. setting the same, the curve is compared with a normal tissue image to determine the attenuation, therefore the hardness. When plotting the tissue amplitude curve along the depth, the tissue with high attenuation will drop with a higher slope.

The conventional ultrasound system assumes the phase velocity in the soft tissue is constant at 1540 m/s, however in the soft organ, liver for instance, when the homogeneous tissue becomes harder, e.g. cirrhosis, the phase velocity in the liver tissue changes, therefore, the assuming of 1540 m/s is no longer valid. The consequences of such assumptions lead to blurred images due to the inaccurate of the beam focusing and the deviations of the geometry distance measurements. The degradation of the image quality might not be considered as significant in the past due to the facts that the conventional image scanning system did not require such image quality and furthermore, it was not expected that the scanned images can achieve consistent quality of sharp and clear images anyway. The ultrasound phase velocity measurement is easier done with the tissue cut out from the body, but extremely difficult in the live organ. For these reasons, it is not obvious to a person of ordinary skill to apply measurement parameters other than the attenuation for hardness characterizations.

In U.S. Pat. No. 6,285,904, Web et al. disclose a method for determining characteristic of tissue that includes steps of supplying optical energy to a tissue and detecting at a plurality of locations consequent energy scattered by the tissue. Analysis of the scattered energy as provides information concerning the properties of the tissue, specifically information related to the fat and lean content and thickness of the tissue. The system includes a light source to deliver optical energy to a tissue and detectors mounted at different positions relative to the source to detect energy scattered by the tissue. A signal processor is used to determine the signal characteristics of the tissue from the detectors and locations of the detectors, specifically information related to the fat and lean content and thickness of the tissue. The patented technique is directed to a method by supplying an optical energy to a tissue where the optical energy has a fixed set of characteristics such as optical projection with fixed wavelength, phase velocity and intensity as that projected from the light source. As discussed above, the measurement accuracies are limited due to this single set of characteristics projected from a single light source operated without controllable input to change the output from the light source.

In U.S. Pat. No. 6,287,259, Grunwald discloses a method to characterize an ultrasonic image of an object by taking into account different in-vivo object parameters and variability of these parameters within a set of ultrasonic image data. Specifically, it is assumed that the object may be defined in terms of statistical properties (or object identifying parameters), which are consistently different from properties of the environment. Such properties are referred to as the object's signature. The statistical properties are calculated at selected locations within the image to determine if they fall within a predetermined range of values which represents the object. If within the range, the locations are marked to indicate they are positioned within the object. A border may then be drawn around the object and the area calculated. Such methodology may be useful for some image analyses applications but would not provide practical solution to overcome the difficulties of insufficient accuracy in measuring and mapping the hardness profile.

In U.S. Pat. No. 6,322,511, a method and apparatus for ultrasound image quantification are disclosed. In U.S. Pat. No. 6,511,430, a process to detect and monitor the process of apoptosis in living tissues by using high frequency ultra sound image systems is disclosed. However, such disclosures do not provide effective methods for a person of ordinary skill to over the problem that accuracy of hardness measurements cannot be easily improved by applying convention techniques as now available.

For these reasons, a need still exists for those of ordinary skill in the art to provide an improved method and system for ultrasonic imaging. Specifically, it is desirable that the ultrasonic imaging system and methods are carried out with more clearly defined and controllable operational parameters with definite functional relationship with hardness measurements such that the inaccuracy and uncertainties of data analyses can be substantially eliminated.

### SUMMARY OF THE INVENTION

It is an object of the present invention to provide a new and more accurate tissue hardness measurement method and system by applying an ultrasound energy to acquire the ultrasonic images, and with the changes of phase velocity in the beam forming calculation until the best focusing image is acquired. The phase velocity with the best-focused image will represent the ultrasound phase velocity in the tissue, and therefore the hardness can be interpolated, and the above discussed difficulties and limitations can be resolved. The criteria of the best focusing image can be an operator decision, or a computer aided estimation, such as the maximum gradient calculation.

In another aspect, the present invention provides an ultrasonic image scanning using the present invention and the conventional amplitude attenuation information together for the tissue hardness estimation.

In yet another aspect, the present invention further provides an ultrasonic imaging scanning system that is implemented to allow an operator's adjustment for the best image focusing estimation controlled by the operator. Additional flexibility may also provide that enables a computer's algorithm for automatic best image searching.

In yet another aspect, the present invention provides a method of constructing a non-linear mapping table to translate the phase velocity into various tissue hardness indexes. Alternate embodiments may implement a color-coded B-mode image according to the different tissue hardness. Furthermore, this invention enables the use of the new phase velocity for the geometry distance and area calculation or applying the same concept for the 3D volume images.

These and other objects and advantages of the present invention will no doubt become obvious to those of ordinary skill in the art after having read the following detailed description of the preferred embodiment, which is illustrated in the various drawing figures.

### BRIEF DESCRIPTION OF FIGURES

The present invention is described in detail below with reference to the following Figures.

FIGS. 1A and 1B, are images of echo signals as illustrations of a prior art scanning system.

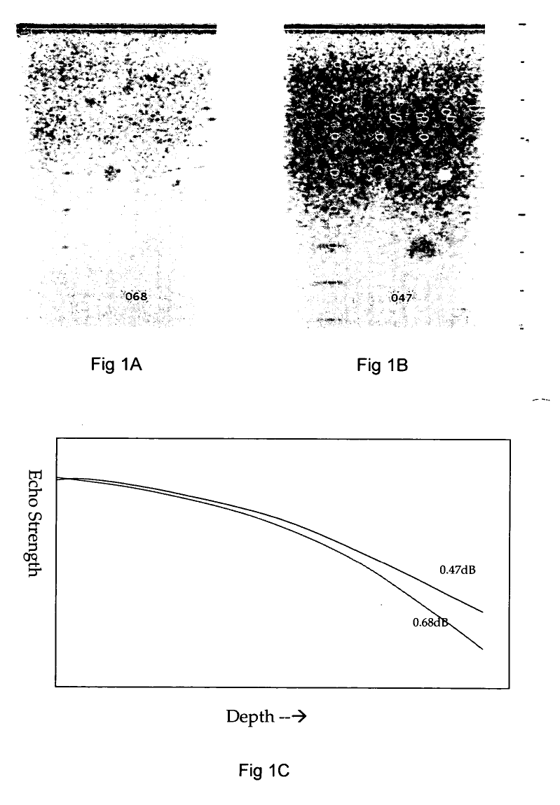

FIG. 1C is a diagram to show the echo strength as function of the depth reflected from the tissue as function of tissue hardness.

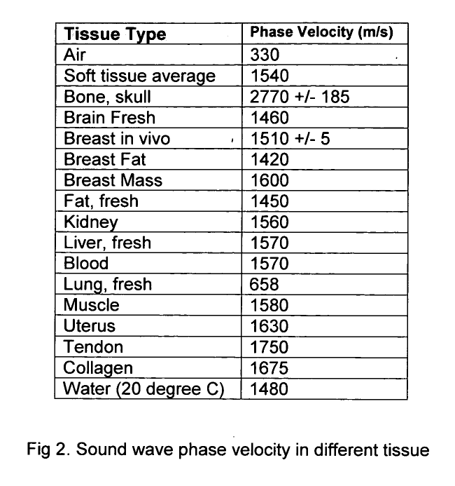

FIG. 2 is a table that shows the changes of phase velocity of as the sound travels in live tissue of different densities and the higher the phase velocity the higher the density of the tissue.

FIG. 3 is a functional block diagram for showing an ultrasound image scanning system with user controllable phase velocity adjustments according to the disclosures of this invention.

FIG. 4 is a diagram for illustrating a beam forming analysis to show how the phase velocity is involved in the beam former calculation and therefore affects the image focusing in the electronics digital beam forming system.

FIG. 5 is a flowchart for showing a process implemented in this invention to obtain an optimal measurement of hardness variations within a soft tissue by applying a user controllable phase velocity adjustment as shown in FIG. 3.

FIGS. 6A to 6D are scanned images obtained by adjusting the phase velocity to illustrate the improvements of hardness measurement accuracy achieved by systems and methods disclosed in this invention.

### DESCRIPTION OF PREFERRED EMBODIMENTS

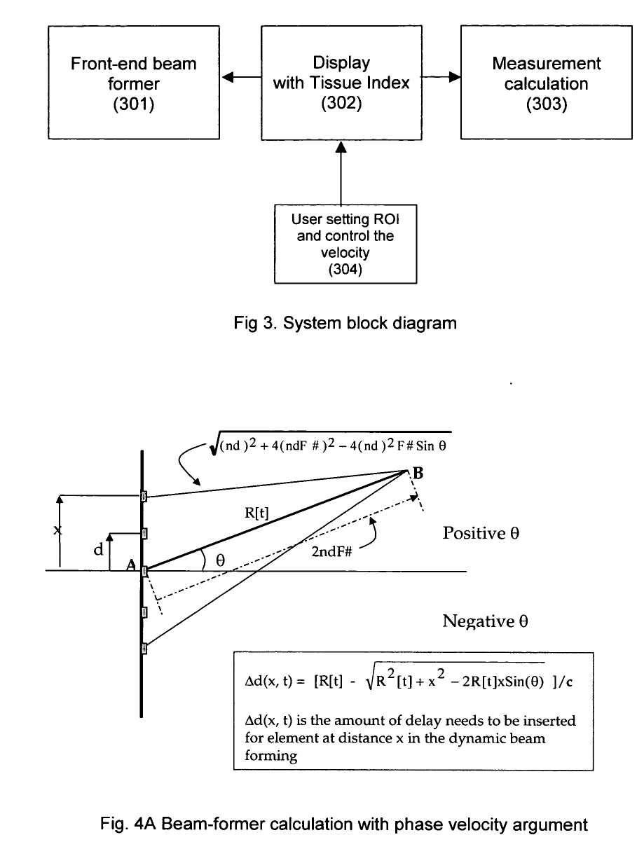

Referring to FIG. 2 for a table that lists media of different tissues for transmission of sound waves. Listed in the table on the right hand column in the table is also the corresponding phase velocity for each of these tissues. According to the listed data shown in FIG. 2, the sound travels in the media, and the phase velocity changes. Depending on the density of the media, the phase velocity varies. As the density of a media increases, the phase velocity of the sound waves transmitted in the media is also increased. Because of these changes, the phase velocity is employed as practical and useful measuring parameter to measure the variations of hardness in a soft tissue. However, the amplitude attenuation may or may not have direct relationship to the tissue hardness. It depends on the tissue homogeneity and absorption.

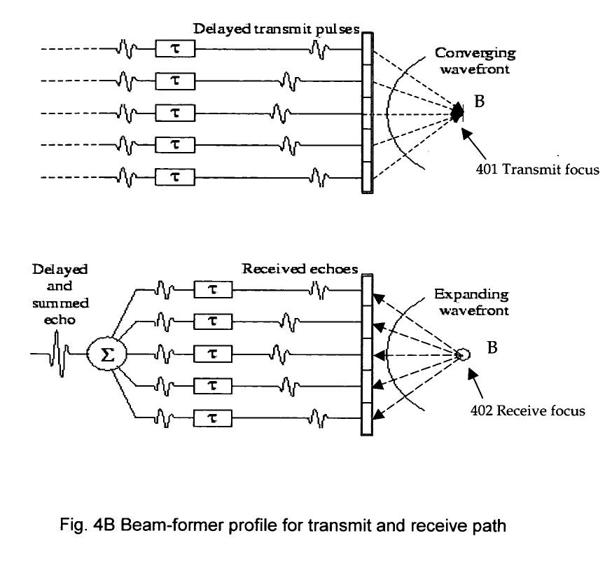

FIG. 3 is a functional block diagram for showing an ultrasound image scanning system implemented with user control functions to adjust the phase velocity to perform an image scan to detect variations of hardness in a soft tissue. The ultrasound image scanning system includes a front-end beam former control circuit 301. A beam profile is first calculated using Equation (4) implemented in a software program as will be further discussed below. The beam profile is a function of the scan line geometrical position and focal point. The results of the calculations will be downloaded into the memory of the beam former 301. It should be noticed that the equation has a parameter C as the phase velocity. As the phase velocity in a tissue changes, then the beam profile will also be changed accordingly and the beam former 301 generates different profiles for different tissues with different phase velocities in the tissues. The image of echo signals from tissue scanning operation is displayed in the image display device 302. A user control unit 304 is provided for a user to change the phase velocity as a controllable parameter in forming the beam while viewing a display images. An operator can adjust the phase velocity in real time through the user interface unit 304 until a sharp image is achieved as that displayed in the display device 302 when the velocity setting matches with the tissue characteristics. A measurement calculation processor 303 carries out a hardness measurement calculation by using the phase velocity to map the round trip echo time into distance. The measurement calculation processor 303 then calculates a hardness profile according to signals received from points located in the tissue at different distances and areas.

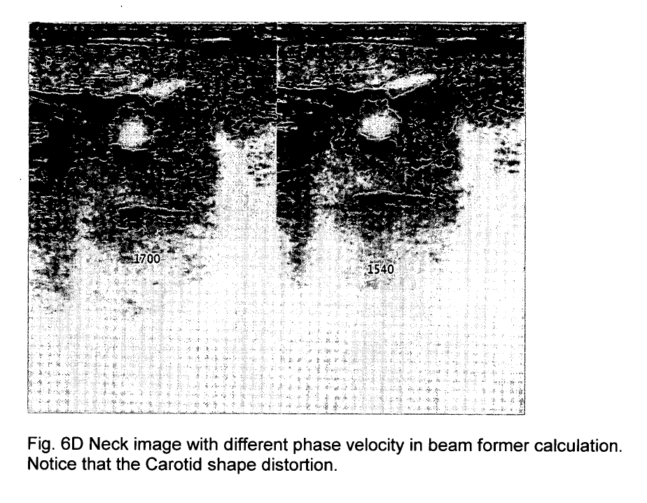

The beam former profile has separate control for beam profile of transmission and beam profile control of reception. By controlling the transmit time delay, all sound waves bursting from the elements are lining up at the focal area and create a constructive enhancement. For receiving the echo signals, the echo signals received by each transducer element are received as signals with a delay profile for the purpose of forming the beam focused through the optical lens. With the advanced digital beam-forming scheme, the beam profile received from the transducer elements can be changed dynamically in real time during the time of reception. Furthermore, the beam can have multiple focal points and change dynamically during the time as the echo signals are transmitted to the ultrasound transducers. The development of beam-formation technologies has made significantly progresses over the past decade such that a highly accurate digital beam-former with progressive dynamic reception focuses can be practically implemented. By employing such digital controllable beam-former, a user is enabled to control the beam formation process to make fine adjustment to the phase velocity of an ultrasound wave. This new capability to control and adjust phase velocity in an ultrasound image system provide significant advantages for image scanning because an analysis illustrates that a slight change in the phase velocity during the beam-forming calculation leads to significant changes in dynamic focus point. Such phase velocity changes can cause dramatic focus changes in the scanned images as clearly illustrated in FIGS. 6A, 6B, and 6C. In addition to the results of measurements, the effects and benefits of controlling and adjusting phase velocity in hardness measurements by applying ultrasound-scanning technologies are further illustrated in the analyses below.

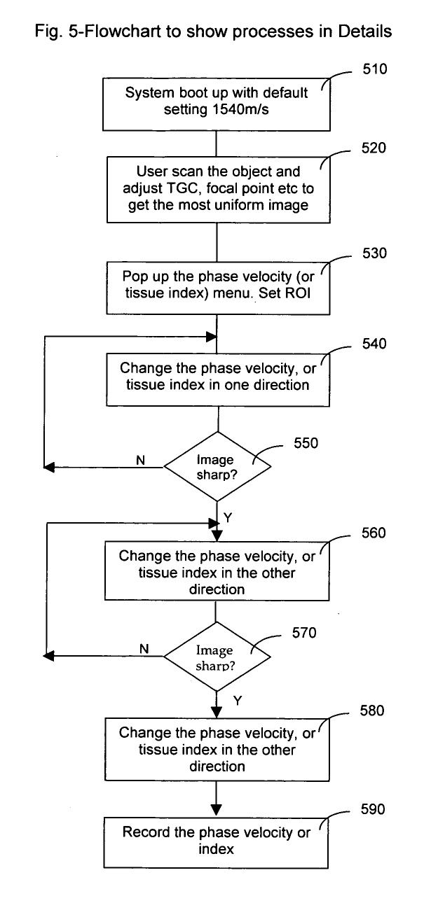

Referring to FIG. 4A for a beam formation calculation. The intersection point A of the horizontal and vertical axes represents the center of the aperture of an ultrasonic transducer element and d is the element pitch distance and point B is the expected focal point at 2D space. FIG. 4B is a diagram for illustrating the concept of signal transmission and reception with delay profiles related to geometries for focusing the beam to obtain sharp and clear images. In 401, the transmitters burst waveform passes through an array of delay control circuits implemented by FIFO (first in first out buffer for example) with separate read and write clock represented by τ₁, τ₂, τ₃, τ₄, before it reaches to the transducer element 405 and emit the energy to the body. Due to this delay profile controlled by the delay circuits represented by τₙ, the burst waveforms from each element will all reach point B at the same time and create a constructive focal energy. In 402, the reflected echo from point B will reach each element at different time. With different delay represented by delay circuit τ′₁, τ′₂, τ′₃, τ′₄ for each element on the receive path before summing them together, the image for one line can be formed. With these delay circuits, the beam former is provided with programmable capability for linear array, curve array and phase array probe modality. In order to control the beam former and the FIFO read and write clocks, there is a memory in the beam former controller to hold the control bits, and the controller downloads these control bits to the delay circuits to enable the control of different delay profiles. With different probes, the profile can be flexibly adjusted and programmed differently.

In order for the waves to focus at point B, the time delay profile relative to point A for each element x is calculated with an equation:
Δd(x,t)=[R[t]−\sqrt{R²[t]+x²−2R[t]×Sin(θ)]}/c
Δd(x,t) is the amount of delay needs to be inserted for element at distance x in the dynamic beam forming.
C is the phase velocity.
R(t) represents the distance of wave propagation at time t
   As shown in the equation, the delay time is a function of the phase velocity C. When the phase velocity changed, the focal point will also be changed. When the phase velocity matches an ultrasound wave phase velocity in the tissue, the wave fronts from every element will reach point B in-phase and form a tight constructive energy spot thus reflects coherent echo signals for measurement as a sharp and clear image. However, when the phase velocity applied by the beam former 301 to project an ultrasound wave into a tissue doesn't match the tissue phase velocity, the ultrasound wave propagation will not have all burst waveforms line up together and become defocused and do not converge to a focal point. Therefore, the functional relationship between the focus profile of a scan image and phase velocity and furthermore, the phase velocity has a functional relationship with the tissue hardness. These functional relationships can therefore be applied to carry out more accurate hardness measurement.

By taking advantage of the correlation as shown in the above equation, a doctor can specify a region of interesting (ROI), i.e., the point B shown in FIGS. 4A and 4B as the intended focus point, through the user interface 304, and adjust the phase velocity setting until a relatively sharp image is clearly shown on the screen. The phase velocity as set by a doctor or alternately by an ultrasound system operator with the best image focused can be used as a reference for the tissue hardness index. The functional relationship between the tissue hardness index and the phase velocity may or may not be a linear relationship. The tissue hardness index affected not only by the phase velocity but also other parameters such as the intensity attenuation and these effects may be combined together and adjusted by a system operator according the quality of image during the real time operations in final fine adjustment of the tissue hardness index. The phase velocity is a primary parameter that is first obtained from calculation to start the initial hardness index input at the beginning of a hardness scan and profiling operation. An example is that 1600 m/s for a sharpest image in liver may refer to the tissue hardness index of 1.2, which is the combination of the phase velocity and other parameters such as intensity attenuation, TGC gain profile, etc.

A software program with built in intelligence to carry out the adjustments of phase velocity and hardness index relationship automatically to assess the quality of the sharpness of an output image displayed on a display device 302. The functions performs by the user can be carried out automatically by programmatically running through different phase velocity setting and automatically select an optimal velocity that produce an image with best display quality. According to the phase velocity, an analysis is then performed to determine the tissue hardness corresponding to the optimal phase velocity.

As progresses are made in the ultrasound control systems, a digital beam former is connected to a processor serving the function as an analyzer to carry out analytical computations as that presented in the Equation above to provide an initial value of the phase velocity. With connection to a processor provided with analytical computational capabilities, the automatic programmable control implemented with different control algorithms and analyses can be more effectively carried out. An operator of an ultrasound hardness profile scanning system can flexibly enter parameters such as ROI, attenuation parameters, phase velocity, auto-phase velocity scanning and optimal image selection, linear array, curve array and phase array probe modality functions to perform an analytical computation for selecting a set of optimal operation parameters to carry out the hardness profile scans. For selection of best images, the processor that serving the functions as an analyzer may also compare different image parameters to select an image with best image quality. The image quality parameters compared may include the determination of a sharpest gradient along the edges of different images from different phase velocity for effectively determine an optimal phase velocity for generating an image with the best image quality.

An operator of the ultrasound systems of this invention further has the controlling options to combine the adjustments of the phase velocity and amplitude attenuation. One example of such control functions can be achieved through entering different parameters into the processor serving the functions as an analyzer. For instance, an operator may take the tissue hardness index out of the phase velocity calculation Hv, and take the tissue hardness obtained from the conventional attenuation method Ha, then applying different weighting to have a final index Ht=w1*Hv+w2*Ha. W1 and W2 are the experiment numbers. With the analytical capabilities provided by the analyzer connected to the beam former, effective control of the beam former with flexible adjustments of measurement parameters is achieved.

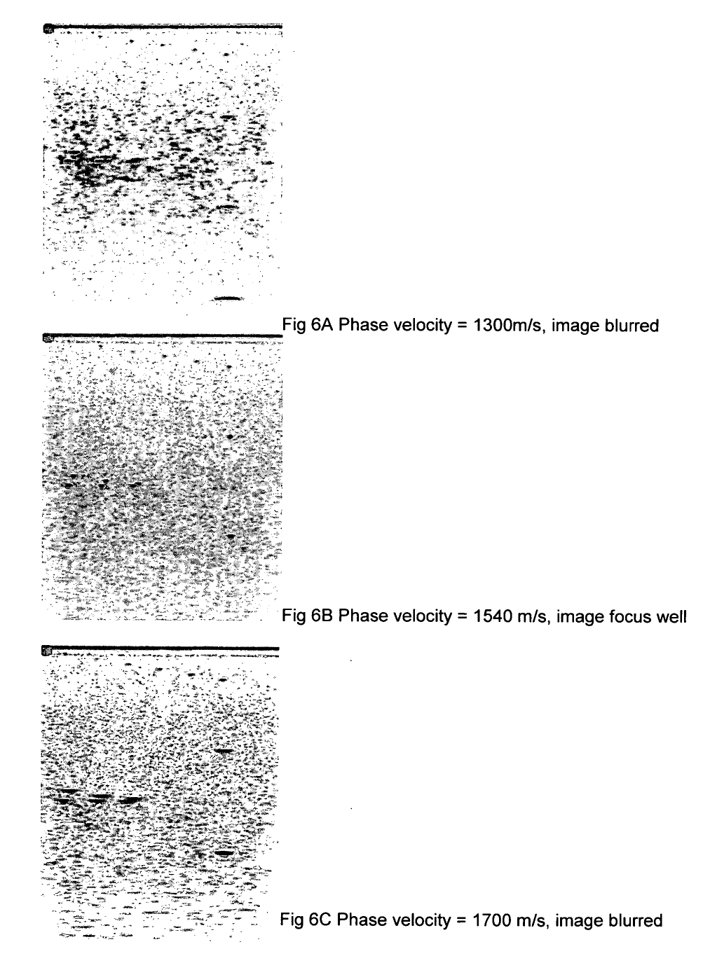

FIG. 5 is a flowchart to show the process for making adjustments for optimizing the tissue hardness index. Initially, system boots up with 1540 m/s phase velocity as default. The process then proceeds with the user adjusts the phase velocity in both directions until the sharpest image is created on the screen as described below. The setting gives the best match with the phase velocity in the specific tissue, and the distance is also correctly measured according to the delay profile analyses as provide by descriptions shown in FIGS. 4A and 4B. The phase velocity as a primary adjustable parameter is now taken into account in the beam forming profile calculation to reduce the error of the geometry measurement.

When system boots-up, the beam forming calculation uses 1540 m/s phase velocity as default (step 510). The user then scans the B-image for the organ, e.g. liver, and adjusts the TGC and focal point based on a ROI to a specific area to obtain a most uniform image (step 520). Then the phase velocity or tissue index soft-menu with options to adjust ROI is popped up in a graphic user interface (GUI) (step 520). With all the TGC curve and gain optimized, the user can change the tissue index setting (step 530), in which the different phase velocities are used for the beam forming calculation (step 540) until the sharpest image is acquired for the ROI (step 550). The index is recorded and can be used for the tissue hardness index. Similar steps as shown in steps 560 to 590 for performing scans in other directions of a same organ is carried out to obtain a better and more complete profiles of the hardness in an organ.

The software can also scan through different phase velocities, and acquire the image, calculate the 2D gradient, sum all the absolute value of the gradient together. With the sharpest image, the total gradient value should be the biggest, and therefore the optimal phase velocity it must be, and the tissue hardness index can be defined.

In the human body, the tissue may have layers of different densities (fat for instance) with different phase velocities, therefore the wave will also cause deflection, refraction and affect the focusing to the under layers. With the phase velocity adjustment, the focusing can be optimized at a specific ROI.

FIGS. 6A to 6C are images obtained by applying different phase velocities (1300 m/s and 1700 m/s) for image scanning experiments with different ultrasound phantoms, and demonstrate the focus changing. FIG. 6D shows that at 1.5 cm depth carotid for neck scan, with the assumption phase velocity error of 160 m/s off 1540 m/s, it can cause carotid diameter measurement error of 5.5% in addition to the image blurring. Referring to FIGS. 6A and 6B, in the measurements are obtained with an assumption that the phase velocity is 1300 m/s while the true value is 1540 m/s. The lateral width measurement of target X will increase from 0.5 mm to 1.0 mm. Same as for the 6B and 6C, the measurement error is 1.5 mm versus 0.5 mm.

According to above descriptions, this invention discloses a method for applying an ultrasound energy to acquire an ultrasonic image. The method includes a step of changing a phase velocity in a beam forming process until a best-focused image is acquired. The method further includes a step of applying the phase velocity with the best-focused image to represent an ultrasound phase velocity in a tissue for interpolating a tissue hardness estimation. In a preferred embodiment, the step of determining the best-focused image includes a step of determining the best-focused image by an operator. In a preferred embodiment, the step of determining the best-focused image includes a step of determining the best-focused image by a computer-aided estimation. In a preferred embodiment, the step of determining the best-focused image includes a step of determining the best-focused image by a computer aided estimation with a maximum gradient calculation. In a preferred embodiment, the step of interpolating the tissue hardness estimation further includes a step of applying the ultrasound phase velocity in the tissue and an attenuation in the tissue for performing a tissue hardness estimation. In a preferred embodiment, the step of determining the best-focused image includes a step of applying an algorithm in a processor connected to a beam former for searching and determining the best-focused image. In a preferred embodiment, the step of interpolating the tissue hardness estimation further includes a step of constructing a nonlinear mapping table to translate the phase velocity into a plurality of tissue hardness indexes. In a preferred embodiment, the step of translating the phase velocity into a plurality of tissue hardness indexes further includes a step of implementing a color-coded B-mode image representing the plurality of different hardness indexes. In a preferred embodiment, the method further includes a step of applying the phase velocity for a geometry distance and area calculation for carrying out a three-dimensional hardness image. In a preferred embodiment, the step of determining the best-focused image further includes a step of determining the best-focused image by a digital controller for providing a real time programmable control.

Again, it is to be understood that the embodiments described are for the purpose of illustration and are not intended to limit the invention specifically to those embodiments. The description and the drawings of the present document describe examples of embodiment(s) of the present invention and also describe some exemplary optional feature(s) and/or alternative embodiment(s). It will be understood that the embodiments described are for the purpose of illustration and are not intended to limit the invention specifically to those embodiments. Rather, the invention is intended to cover all that is included within the spirit and scope of the invention, including alternatives, variations, modifications, equivalents, and the like.

I claim:

1. An ultrasonic image scanning system for scanning an organic object comprising:
a beam former includes a phase velocity adjustment function for producing an ultrasonic image with a programmable phase velocity.

2. The ultrasonic image scanning system of claim 1 further comprising:
a beam profile analysis function for calculating an optimal phase velocity.

3. The ultrasonic image scanning system of claim 1 further comprising:
a user controller for a user of said ultrasonic image scanning system to adjust said phase velocity.

4. The ultrasonic image scanning system of claim 1 further comprising:
an automatic phase velocity scanning controller for automatically scanning through a range of phase velocities and selecting a best phase velocity generating a scanning image of a best quality.

5. The ultrasonic image scanning system of claim 1 further comprising:
a region of interest (ROI) controller for a user to select a region for scanning with a specific focal area for optimizing said phase velocity.

6. The ultrasonic image scanning system of claim 1 wherein:
said beam former further comprising a maximum gradient analyzer for selecting an image of a best quality in optimizing said phase velocity.

7. The ultrasonic image scanning system of claim 1 wherein:
said beam former further comprising a digital controller for providing a real time programmable control.

8. The ultrasonic image scanning system of claim 1 wherein:
said beam former further comprising a digital controller for providing a real time linear-array programmable control.

9. The ultrasonic image scanning system of claim 1 wherein:
said beam former further comprising a digital controller for providing a real time curve-linear-array programmable control.

10. The ultrasonic image scanning system of claim 1 wherein:
said beam former further comprising a digital controller for providing a real time phase-array-probe programmable control.

11. The ultrasonic image scanning system of claim 1 wherein:
said beam former further comprising a phase velocity and attenuation adjustment controller for producing said ultrasonic image with an adjustable phase velocity and amplitude attenuation.

12. The ultrasonic image scanning system of claim 1 further comprising:
said beam former further includes a hardness computational processor for determining a tissue hardness using said phase velocity in combination with an attenuation parameter.

13. A method for applying an ultrasound energy to acquire an ultrasonic image comprising:
changing a phase velocity in a beam forming process until a best-focused image is acquired.

14. The method of claim 13 further comprising:
applying said phase velocity with said best-focused image to represent an ultrasound phase velocity in a tissue for interpolating a tissue hardness estimation.

15. The method of claim 13 further wherein:
said step of determining said best-focused image comprising a step of determining said best-focused image by an operator.

16. The method of claim 13 further wherein:
said step of determining said best-focused image comprising a step of determining said best-focused image by a computer aided estimation.

17. The method of claim 13 further wherein:
said step of determining said best-focused image comprising a step of determining said best-focused image by a computer aided estimation with a maximum gradient calculation.

18. The method of claim 14 wherein:
said step of interpolating said tissue hardness estimation further comprising a step of applying said ultrasound phase velocity in said tissue and an attenuation in said tissue for performing a tissue hardness estimation.

19. The method of claim 13 further wherein:
said step of determining said best-focused image comprising a step of applying an algorithm in a processor connected to a beam former for searching and determining said best-focused image.

20. The method of claim 14 wherein:
said step of interpolating said tissue hardness estimation further comprising a step of constructing a nonlinear mapping table to translate said phase velocity into a plurality of tissue hardness indexes.

21. The method of claim 20 wherein:
said step of translating said phase velocity into a plurality of tissue hardness indexes further comprising a step of implementing a color-coded B-mode image representing said plurality of different hardness indexes.

22. The method of claim 14 further comprising:
applying said phase velocity for a geometry distance and area calculation for carrying out a three-dimensional hardness image.

23. The method of claim 13 wherein:
said step of determining said best-focused image comprising a step of determining said best-focused image by a digital controller for providing a real time programmable control.
